(19) United States
(12) Patent Application Publication
(10) Pub. No.: US 2026/0026776 A1
(43) Pub. Date: Jan. 29, 2026

(54) BLOOD FLOW MONITORING

(71) Applicant: Flow RT, LLC, Boerne, TX (US)

(72) Inventors: Oscar Ochoa, Boerne, TX (US); Jun Yang, Boerne, TX (US)

(73) Assignee: Flow RT, LLC, Boerne, TX (US)

(21) Appl. No.: 19/033,127

(22) Filed: Jan. 21, 2025

Related U.S. Application Data
(60) Provisional application No. 63/675,608, filed on Jul. 25, 2024.

(51) Int. Cl. A61B 8/06; A61B 8/00; A61B 8/12

(52) U.S. Cl. CPC A61B 8/06; A61B 8/12; A61B 8/4472; A61B 8/488; A61B 8/54; A61B 8/56

(57) ABSTRACT

Methods and systems described herein may be configured for receiving a doppler shifted signal from a probe installed at least partially in a free tissue flap; storing the doppler shifted signal in an integrated storage device or uploading the doppler shifted signal in a one or more networks of remote servers. Further, they may comprise processing at least part of the doppler shifted signal on the integrated storage device or on the one or more networks of remote servers with a signal processor to form a blood flow sample of the free tissue flap; and determining a qualitative blood flow status or a quantitative blood flow status of the free tissue flap with at least one blood flow sample. In addition, methods and systems may comprise a transceiver, a signal processor; a controller; and a wireless module.

### BACKGROUND

The present invention relates to blood flow monitoring, and, more particularly, but not by way of limitation, to a blood flow monitor configured to post-operatively monitor patient blood flow. Through signal processing, described herein, data acquisition and analysis, surgery specific and systemic patient hemodynamic monitoring are enabled. In response to the blood flow monitor determining that surgery specific patient blood flow is abnormal, output a signal notifying of the abnormal blood flow, including wirelessly communicating with a clinician.

Reconstructive surgery is an integral field in medical care secondary to patients commonly experiencing soft tissue defects from conditions including, but not limited to, traumatic tissue loss or cancer extirpation surgery. Free tissue reconstruction entails designing, harvesting, and transferring a segment of the patient's own soft tissue including its source blood vessels (also known as a tissue flap) from a distant site in the patient's body to a site involving a soft tissue defect in order to replace or address the patient's specific needs. Reestablishing and maintaining continuous blood flow within the tissue flap used for reconstruction is absolutely critical, not only for the success of the surgical procedure, but also to minimize complications. Reestablishment of continuous blood flow requires blood vessel (arteries and veins) connections, known as anastomoses, to be made between the recipient soft tissue defect site and source blood vessels contained within the tissue flap harvested for reconstruction. These vascular anastomoses are performed by hand commonly using a high-powered microscope, hence the term “microvascular reconstruction” utilized for this surgical specialty. Due to multiple factors, including the patient's overall hemodynamic status and the small size of blood vessels, vascular anastomoses are extremely delicate and prone to occlusion during the first five days of the immediate post-operative period. Thorough and consistent post-operative monitoring may be necessary for identifying a patient's post-operative hemodynamic condition. This allows for managing a patient's condition including rectifying episodes of vascular occlusion associated with the reconstructive tissue in a timely fashion before irreversible tissue damage occurs. Current post-operative screening methods for assessing the health of the reconstructive tissue are: 1) physical exams performed by specially trained medical professionals and, 2) blood flow tissue sensors.

Unfortunately, both the above-listed screening methods have significant limitations in the current health care landscape. To illustrate, physical examination is very time and labor intensive, often requiring intensive care unit (ICU) admission to receive an appropriate level of nursing oversight. Thus, adding to the significant overall resource and financial cost associated with free tissue reconstruction procedures. In addition, while blood flow sensors are continuous and often facilitate identifying actionable events earlier than physical exam findings, they have limitations. For example, current blood flow sensors do not assess systemic overall patient hemodynamic status and, importantly, require the patient be tethered to bedside equipment due to their wired design.

Tethering patients to bedside equipment severely impedes early patient mobilization, which has been shown to expedite patient recovery and reduce the incidence of venous thromboembolism. In fact, during the immediate post-operative period, length of hospital stay is primarily determined, not by overall patient condition as in conventional surgery, but more so by the blood flow monitoring needs related to the reconstructive procedure. As such, potential patient discharge is currently protracted despite meeting clinical criteria if tissue blood flow monitoring is still desired. Accordingly, a blood flow monitor that uniquely allows post-operative patient mobilization while maintaining thorough assessment of systemic hemodynamic status is desired.

### BRIEF DESCRIPTION OF THE DRAWINGS

FIG. 1A is a schematic diagram illustrating a blood flow monitor and the connection thereof with a blood vessel via a probe.

FIG. 1B is a venous coupler probe.

FIG. 1C is a schematic diagram illustrating how a venous coupler probe is connected to a blood vessel.

FIG. 1D is a blood vessel being actively measures by a venous coupler probe.

FIG. 2 is a schematic diagram illustrating the blood flow monitor and an example securing thereof with a patient.

FIG. 3 is a block diagram illustrating the blood flow monitor and the connection thereof with the probe and a remote notification device.

FIGS. 4-10 are flow diagrams illustrating operation of the blood flow monitor in monitoring blood flow through a blood vessel.

FIG. 11 is a graph illustrating an example waveform of a blood flow signal.

FIG. 12 is a graph illustrating an example energy waveform developed from energy sample values taken from the example blood flow signal shown in FIG. 11.

### DETAILED DESCRIPTION

Methods and systems for blood flow monitoring are described below. Specifically, methods and systems may comprise continuously monitoring tissue blood flow within the reconstructive free flap without tethering patients while broadening professional oversight capabilities. In addition, the ability for continuously monitoring in an outpatient setting enables expedited safe patient discharge. This may reduce healthcare costs and improve hospital resource utilization. To perform this, blood flow monitoring may compare the peaks, dips, and status of waveforms that comprise blood flow, energy, power, and reference signals over a set amount of time. Data is obtained in a complex algorithm that incorporates a counting and averaging system. The data is processed, amplified, and filtered for digitalization. Through data processing and digitalization, local measurements at the free tissue flap comprising blood flow waveforms are generated and analyzed to determine the blood flow status of the corresponding reconstructive free tissue flap. A free tissue flap is a surgical procedure in which a section of tissue, along with its blood supply, is completely detached from one part of the body and then transferred to another location. This technique is commonly used in reconstructive surgeries to restore form and function. For the tissue to successfully integrate at its new location, it needs sufficient blood flow.

Monitoring the blood flow at the new location is essential because medical staff must be alerted if there is not sufficient blood flow. Systems and methods described below may calculate the blood flow volume within one or more vessels of the free tissue flap. For example, blood flow monitors described below may enable the generation of a waveforms. The waveform is a graph of tabulated data. Through one or more filters, the waveforms may be analyzed and in connection with a vascular probe to calculate the blood flow, to be discussed in detail below. With blood flow volume, qualitative and quantitative values of a patient's blood flow status may be determined.

Qualitative blood flow status may be determined by whether or not the waveform indicates there is blood flow within a blood vessel. Quantitative blood flow status values may be computed utilizing a blood flow conversion factor. Quantitative blood flow status may be systematic blood flow throughout the body. In examples, quantitative blood flow may be the total cardiac output, blood flow within specific regions of the body, and/or computed products of systemic patient hemodynamic status in combination with blood flow. Both quantitative and qualitative blood flow status may be determined and provided to the clinician. A blood flow conversion factor may be a translator of a series of determinants with different calculated values. The conversion factor is the calculated value from the waveform and/or determined on a on a case by case or patient by patient depending on factors of the patient, procedure, new location, origin of free tissue flap and/or the like.

One or more waveforms may be represented as a series of data. Herein, at least part of the blood flow conversion factor may be a measure of how much blood should flow for a specific tissue. The blood flow conversion factor may also be a ratio of the standard/normal/healthy/etc. blood flow through a vessel to the total volume of blood in the body. A blood flow conversion factor may be pre-determined based on the type of tissue at the free tissue flap, or it may be determined at the discretion of medical staff or any algorithm. Tissues with less blood flow, such as tendons may have a low blood flow conversion factor, while tissues with more blood flow, such as critical organs may have a high blood flow conversion factor.

Herein, a healthy threshold may be defined as whether there is enough blood delivered to a free tissue flap. The healthy threshold may be determined by the blood flow status and the blood flow conversion factor. The healthy threshold may be analyzed and determined on a case by case or patient by patient basis. Further, in addition to local measurements, the overall conditions of the patient comprising hemodynamic status may also be monitored and used to determine the healthy threshold. However, not every example requires hemodynamic status. Monitoring may continue of the blood flow status exceeds the healthy threshold, but if it dips below, intervention may be required. Further, artificial intelligence and/or machine learning algorithms may also be applied to process waveforms and/or determine qualitative and quantitative blood flow status based on the waveforms and/or a blood flow conversion factor.

Metrics related to systemic patient hemodynamic status (or systemic blood flow) such as heart rate, cardiac stroke volume, stroke volume variability, and cardiac output may be obtained. The monitoring device may be enabled to transmit data to everyday devices such as a phone, computer, tablet, etc. allowing remote patient monitoring and patient data acquisition. It is to be understood that the disclosed examples are merely exemplary, which may be in various forms. It is further to be understood that the figures are not necessarily to scale, and some features may be exaggerated to show details of particular components or blocks.

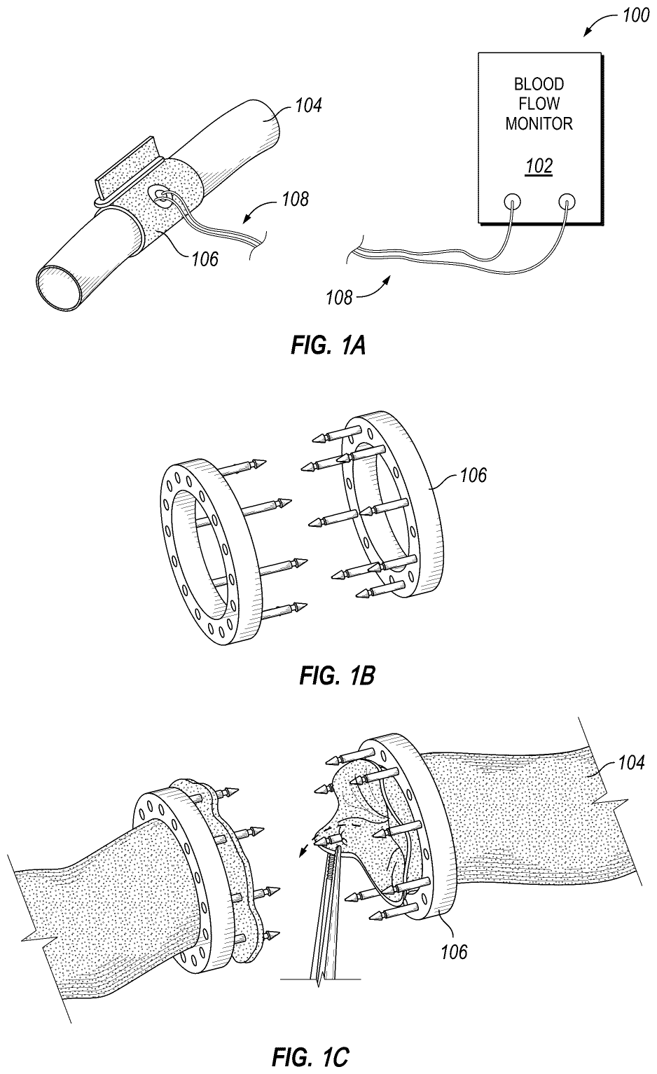

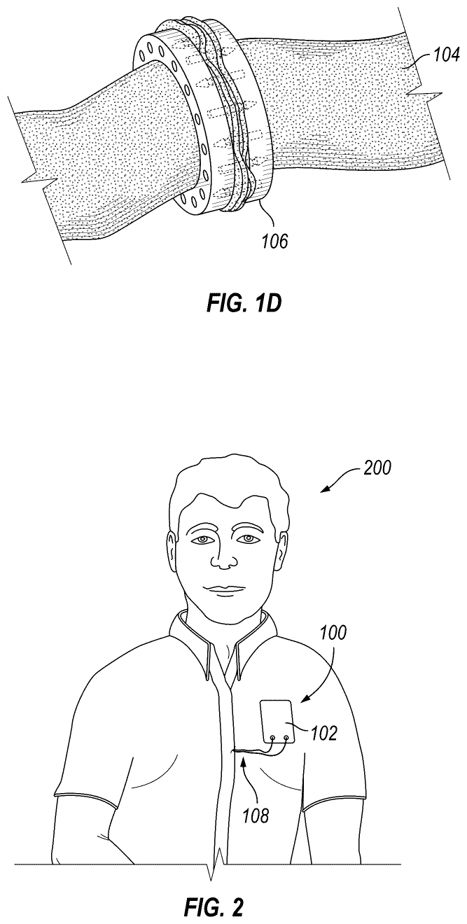

Generally, as an overview, and to be discussed in greater detail below, a blood flow monitor 100 as illustrated in FIGS. 1A and 2 comprises a housing 102 while being configured to post-operatively monitor blood flow of a patient 200 through direct measurement of hemodynamic changes within a blood vessel of interest and calculation of systemic hemodynamic conditions or systemic blood flow. The patient 200, during a reconstructive surgery including correction of a soft tissue defect, experiences a transfer of the patient's own soft tissue including source blood vessels from a distant site in the patient's body to a site involving the soft tissue defect. During a transfer, a piece of tissue of patient 200 may be moved from one location to another. A Transfer may comprise blood vessel (arteries and veins) connections made between the recipient site and source blood vessels within the tissue harvested for reconstruction. Upon completion of the blood vessel connections, patient 200 at connected blood vessels 104 is fitted with a probe 106 used by the blood flow monitor 100 in developing the blood flow wave form and other systemic hemodynamic metrics or systemic blood flow to be discussed herein. The blood flow monitor 100 in the example operatively connects with the probe 106 through a wired connection 108 configured for transmitting signals to the probe 106 with doppler effect capabilities and receiving signals from the probe 106, to be discussed further below. The wired connection 108 operatively connects with both the probe 106 and the blood flow monitor 100 using a removable plug or a hardwired connection.

After completion of the reconstructive surgery, the blood flow monitor 100, which has been operatively connected with probe 106 using the wired connection 108, is wearable by the patient 200. In contrast, current monitors are also connected to the vascular probe but are much larger and prohibit easy patient mobilization and ambulation. In examples, wired connection 108 may be replaced with a wireless connection. Blood flow monitor 100 accordingly allows immediate post-operative patient mobilization and a potential for patient discharge that minimizes not only healthcare costs but also hospital resource utilization. The blood flow monitor 100 in developing the blood flow waveform using the probe 106 operatively connected with the blood vessels 104 outputs a reference signal to the probe 106. The probe 106 after receiving the reference signal applies the reference signal at the blood vessels 104 to produce a doppler shifted signal indicative of blood flow through the blood vessels 104. The probe 106 upon developing the doppler shifted signal returns the doppler shifted signal to the blood flow monitor 100. The blood flow monitor 100 receives the doppler shifted signal and processes the doppler shifted signal into a signal representing velocity of blood flow through the blood vessel 104, hereinafter referred to as a blood flow signal.

The blood flow monitor 100 further generates a patient blood flow waveform using the blood flow signal. The blood flow monitor 100 upon generating the patient blood flow waveform determines through pattern recognition algorithms whether the patient blood flow waveform pattern represents a normal blood flow waveform indicating normal blood flow through the blood vessels 104 or an abnormal blood flow waveform pattern indicating abnormal blood flow through the blood vessels 104. The blood flow monitor 100 in response to the abnormal blood flow waveform pattern outputs an alarm signal to the patient 200 and a wireless alarm signal to a clinician informing the clinician of the abnormal blood flow waveform pattern indicating abnormal blood flow through the blood vessels 104. In addition, with utilization of a vascular probe with a known luminal diameter, blood flow velocity will be utilized to calculate volume of blood flow within the blood vessel producing, through application of a known conversion factor, clinically important systemic hemodynamic or systemic blood flow measurements such as cardiac stroke volume, stroke volume variability and, in conjunction with calculated patient heart rate, cardiac output.

FIG. 1B illustrates a venous coupler as an example of probe 106. FIG. 1C is a schematic diagram illustrating how a venous coupler probe as probe 106 may be connected to blood vessel 104. FIG. 1D illustrates blood vessel 104 being actively measures by a venous coupler probe as probe 106.

The blood flow monitor 100, the configuration and processes thereof which will be further explained in greater detail below, may be implemented in hardware, firmware, software, and combinations of these. The blood flow monitor 100 comprises a single processor or set of processors that in an example, may be any central processing unit (CPU), microprocessor, microcontroller, device or circuit configured for executing computer program instructions. The processor or processors may comprise a timer and clock as well as all the usual ancillary components necessary to form a functional blood flow monitor including without limitation a bus, non-transitory storage such as RAM or ROM containing instructions, input/output devices, user interfaces, removable data storage, and any form of wireless communication interfaces. Any communication standard for wireless communication interfaces may be utilized. Software and data described may be programmed into non-transitory storage memory as instructions that are accessible to and retrievable by a processor or processors as described herein which configures and directs the processor or processors to perform the desired functions and processes through execution of the instructions.

A “signal” is defined herein as a detectable physical quantity or impulse such as a voltage, current, or magnetic field strength by which messages or information may be transmitted, and hereafter is defined to comprise an object used to transmit or convey information. It is understood that the processing of a signal that occurs is done through the interaction of various components such as for example microcontrollers, transceivers, circuits, and antennas.

Terms such as “attached,” “connected,” “coupled,” “interconnected,” and similar refer to a relationship wherein structures are secured or attached to one another either directly or indirectly through intervening structures, as well as both movable or rigid attachments or relationships, unless expressly described otherwise.

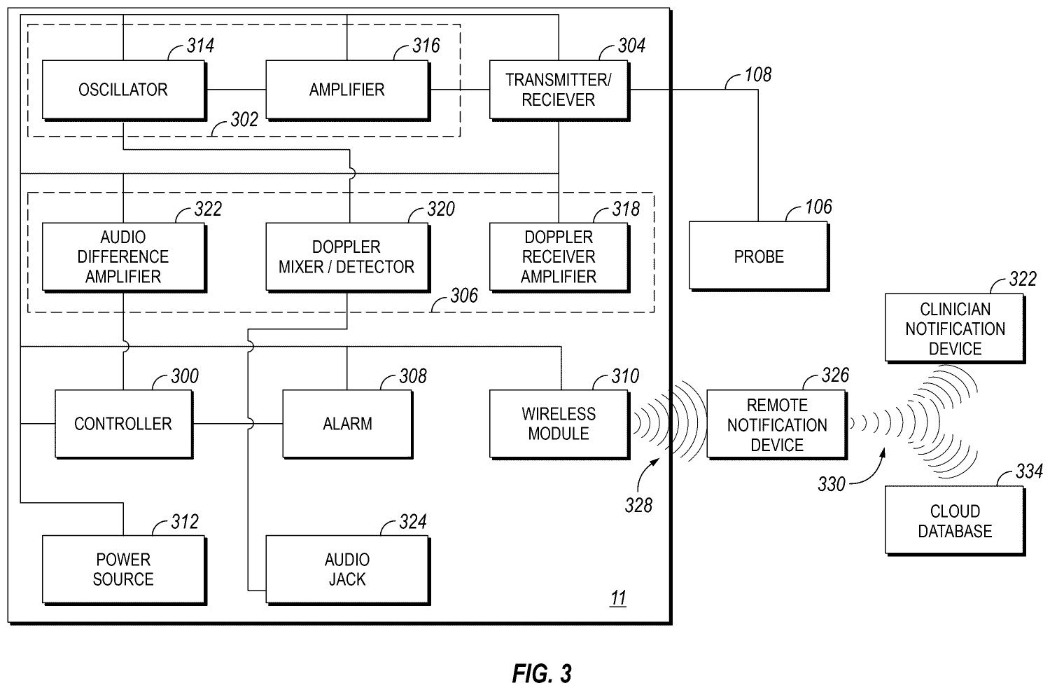

The blood flow monitor 100 as illustrated in FIG. 3 comprises a controller 300 operatively connected with a reference signal generator 302, a transmitter/receiver 304 configured to operatively connect with the probe 106 through the wired connection 108, a signal processor, an alarm 308, and a wireless module 310. In addition to the controller 300, the reference signal generator 302 operatively connects with the transmitter/receiver 304 and the signal processor 306, while the transmitter/receiver 304 operatively connects with the signal processor 306. The controller 300 in an example, is any suitable processor or set of processors that perform basic arithmetic, logic, controlling, timing, clocking, and input/output operations specified by instructions in a program stored within the processor or set of processors or a memory unit associated with the processor or set of processors. The controller 300 as will be described more fully herein controls power applied to the reference signal generator 302, the transmitter/receiver 304, the signal processor 306, the alarm 308, and the wireless module 310. In examples, the wireless module is configured to initiate a buzzer or alarm that alerts patient and/or text message or notification to a clinician. The controller 300 as will be described more fully herein further communicates with and controls the transmitter/receiver 304, the signal processor 306, the alarm 308, and the wireless module 310 during generation of a patient blood flow signal, heart rate value, and alarms as needed.

The blood flow monitor 100 comprises a power source 312 interconnected with the controller 300, the reference signal generator 302, the transmitter/receiver 304, the signal processor 306, the alarm 308, and the wireless module 310 in order to apply a voltage and thus supply power to the controller 300, the reference signal generator 302, the transmitter/receiver 304, the signal processor 306, the alarm 308, and the wireless module 310. While the power source 312 applies a voltage to distribute power throughout the blood flow monitor 100 including to the controller 300, the controller 300 as will be described more fully herein regulates the distribution of power from the power source 312 throughout the blood flow monitor 100 in order to provide the blood flow monitor 100 with a power saving mode. In accordance with the incorporation into the blood flow monitor 100 of the power saving mode, the power source 312 in an example, is a portable power source, such as, for example, a battery, that produces portability in the blood flow monitor 100 through separating the blood flow monitor 100 from a fixed power source, such as a power outlet.

The reference signal generator 302 generates a reference signal, which, in an example, may be an ultrasound signal applied as a voltage having a known frequency of 20 MHz. The reference signal accordingly provides the blood flow monitor 100 with a known frequency signal utilized to drive the probe 106 while further providing a reference used by the blood flow monitor 100 when determining patient blood flow and waveform generation. The reference signal generator 302 in an example, comprises an oscillator 314 operatively connected with an amplifier 316 and with the signal processor 306. The oscillator 314 generates the reference signal which in an example, is a crystal-controlled reference signal. The oscillator 314 outputs the reference signal to the amplifier 316, which, in turn, amplifies the reference signal to a level necessary to drive the probe 106. The oscillator 314 further outputs the reference signal to the signal processor 306 in order to provide a reference used by the signal processor 306 in developing the blood flow signal as will be described more fully herein.

The transmitter/receiver 304 operatively connects with the reference signal generator 302 and more particularly with the amplifier 316 to receive the reference signal from the amplifier 316 and thus the reference signal generator 302. The transmitter/receiver 304 operatively connects with the probe 106 using the wired connection 108 whereby the wired connection 108 secures with both the probe 106 and the transmitter/receiver 304 using a hardwired connection or a removable plug. Moreover, the transmitter/receiver 304 under the control of the controller 300 outputs the reference signal to the probe 106 and then receives from probe 106 using the wired connection 108 a doppler shifted signal indicative of blood flow through the blood vessels 104. The transmitter/receiver 304 further operatively connects with the signal processor 306 whereby, as will be described more fully herein, the transmitter/receiver 304 under the control of the controller 300 outputs the doppler shifted signal to the signal processor 306 for use in developing the blood flow signal.

Once the transmitter/receiver 304 in response to the controller 300 outputs the reference signal to probe 106, it applies the reference signal to the connected blood vessels 104. Probe 106 in an example and any device suitable to produce the doppler shifted signal, such as, for example, a Doppler ultrasound probe, with a venous coupler probe (e.g., referring to FIGS. 1B-1D) with a known diameter of 2.0, 2.5, and 3.0 mm or with a cook probe that is attached using a silicon strip tailored to the variable diameter of the blood vessel 104. In further examples, probe 106 may be any form of a doppler shifted probe or any form of a vascular probe. The reference signal when applied to the blood vessels 104 by the probe 106 traverses the blood vessels 104 and then reflects back to the probe 106, thereby producing the doppler shifted signal. As described above, probe 106 may take varying examples, however, they are small and effective in application. In addition, blood flow monitor 100 may either be very lightweight or completely remote. To be discussed below, its processing may be done remotely or locally. At least all or part of the processing techniques described above may be performed locally and/or remotely. Blood flow monitor 100 may be greatly reduced in size in comparison to surface monitors. Thus, the overall application is lighter and more mobile for patients and clinicians. To illustrate, methods and systems do not require that a patient be tethered to a bed for blood flow monitoring. Instead, probes 106 may be installed within the free tissue flap of a patient. Such probes 106 do not require complex wiring or require the patient to stay in a hospital during recovery. In contrast, current technology relies on surface monitors. These devices may be large and complex requiring wires and difficult installation.

The reference signal in traversing the blood vessels 104 while being reflected to probe 106 experiences the Doppler effect when blood is moving through the blood vessels 104 whereby the reference signal undergoes a shift in frequency that creates the doppler shifted signal. The doppler shifted signal accordingly in an example, is a frequency shifted voltage signal created through the Doppler effect distinct from the reference signal. Moreover, the frequency shift experienced by the reference signal when blood is moving through the blood vessels 104 in order to produce the doppler shifted signal is dependent upon velocity of blood flowing through the blood vessels 104 in that a greater velocity produces a greater Doppler frequency shift such that, as will be described more fully herein, the doppler shifted signal allows the blood flow monitor 100 to develop the blood flow signal. The probe 106 after generation of the doppler shifted signal transmits the doppler shifted signal to the transmitter/receiver 304 for use by the blood flow monitor 100 in generating the blood flow signal.

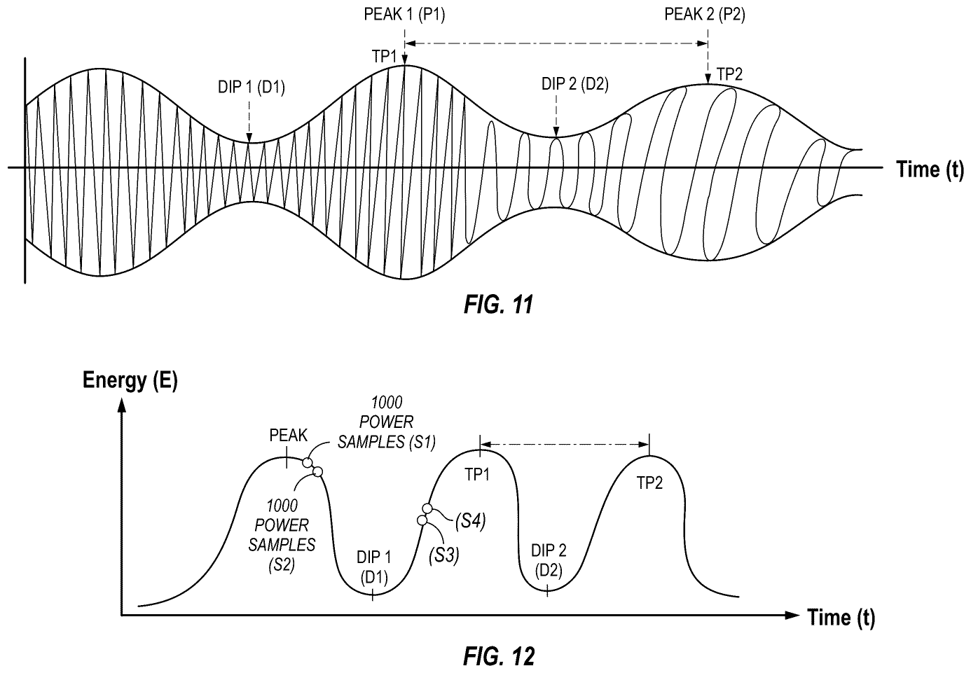

The signal processor 306 operatively connects with the transmitter/receiver 304 to receive the doppler shifted signal from the transmitter/receiver 304 and then process the doppler shifted signal into a blood flow signal. Once the transmitter/receiver 304 receives the doppler shifted signal from the probe 106, the transmitter/receiver 304 under the control of the controller 300 outputs the doppler shifted signal to the signal processor 306. The signal processor 306 after receiving the doppler shifted signal from the transmitter/receiver 304 first amplifies the doppler shifted signal prior to multiplying the reference signal received from the oscillator 314 of the reference signal generator 302 with the doppler shifted signal resulting in the blood flow signal. The blood flow signal in the example illustrated in FIG. 11 is a voltage signal having a frequency that is the difference between the frequency of the reference signal and the frequency of the doppler shifted signal. Since the doppler shifted signal is a frequency shifted signal dependent upon the velocity of the blood flowing through the blood vessels 104, the frequency of the blood flow signal, which is the difference in frequency between the reference signal and the frequency of the doppler shifted signal, is a measure of the velocity of the blood flowing through the blood vessels 104. In accordance therewith, the blood flow signal, as will be described more fully herein, is usable to develop a patient blood flow waveform illustrating fluctuations in blood flow velocity within the source blood vessel 104. In addition to the blood flow signal providing a measure of the velocity of the blood flowing through the blood vessels 104, the blood flow signal in an example, is an audio signal expressed as a voltage with a frequency in the audio range. Upon developing the blood flow signal, the signal processor 306, as will be described more fully herein, amplifies a blood flow sample taken from the blood flow signal to a signal level suitable for digitizing and subsequent use by the controller 300 in generating the patient blood flow waveform.

The signal processor 306 in an example, comprises a doppler receiver amplifier 318, a doppler mixer detector 320 operatively connected with the oscillator 314 of the reference signal generator 302, and an audio difference amplifier 322 operatively connected with the controller 300. The doppler receiver amplifier 318 operatively connects with the transmitter/receiver 304 to receive the doppler shifted signal from the transmitter/receiver 304 and then amplify the doppler shifted signal. The doppler mixer detector 320 operatively connects with the doppler receiver amplifier 318 to receive the doppler shifted signal as amplified and then multiply the reference signal received from the oscillator 314 with the doppler shifted signal in order to produce the blood flow signal. The audio difference amplifier 322 operatively connects with the doppler mixer detector 320 to receive the blood flow signal and then, under the control of the controller 300, amplify a blood flow sample taken from the blood flow signal to a signal level suitable for digitizing and subsequent use by the controller 300.

The controller 300 in an example, while the blood flow monitor 100 is powered on and not in a power saving mode, produces the blood flow signal using the reference signal and the doppler shifted signal. More particularly, the controller 300 creates the doppler shifted signal by intermittently controlling the transmitter/receiver 304 to develop doppler shifted signal samples generated over time measured in seconds, whereby the doppler shifted signal samples form the doppler shifted signal, and then output the doppler shifted signal samples to the signal processor 306 for sequential processing into blood flow signal samples generated over time measured in seconds, whereby the blood flow signal samples form the blood flow signal as expressed by the example waveform of a blood flow signal shown in FIG. 11. Illustratively, the controller 300 controls the transmitter/receiver 304 to receive the reference signal from the reference signal generator 302 and more particularly the amplifier 316 and then output the reference signal to the probe 106. The probe 106 upon receiving the reference signal generates a doppler shifted signal sample indicative of blood flow through the blood vessels 104. After the transmitter/receiver 304 outputs the reference signal to the probe 106, the controller 300 controls the transmitter/receiver 304 to receive the doppler shifted signal sample from the probe 106 and then output the doppler shifted signal sample to the signal processor 306 and more particularly the doppler receiver amplifier 318. Upon receipt of the doppler shifted signal sample, the doppler receiver amplifier 318 amplifies the doppler shifted signal sample prior to outputting the doppler shifted signal sample as amplified to the doppler mixer detector 320. The doppler mixer detector 320 receives the doppler shifted signal sample as amplified from the doppler receiver amplifier 318 and the reference signal from the oscillator 314 whereby the doppler mixer detector 320 multiplies the reference signal with the doppler shifted signal sample in order to produce a blood flow signal sample. Once the transmitter/receiver 304 outputs the doppler shifted signal sample to the signal processor 306, the controller 300 controls the transmitter/receiver 304 to receive another doppler shifted signal sample and then output the doppler shifted signal sample to the signal processor 306 for processing into another blood flow signal sample. The controller 300 accordingly controls the transmitter/receiver 304 to sequentially develop doppler shifted signal samples and then output the doppler shifted signal samples to the signal processor 306 for processing into blood flow signal samples that form the blood flow signal shown in the example blood flow signal waveform of FIG. 11. The controller 300 intermittently controls the transmitter/receiver 304 in the development of the blood flow signal until the blood flow monitor 100 enters the power saving mode thereof.

The generated waveform, representing sequential blood flow signals, will create a characteristic oscillating contour pattern (FIG. 11) under normal blood flow conditions. However, under conditions where blood flow through the vessel is compromised by extrinsic compression (operative site hematoma or vessel kinking), intrinsic occlusion (venous thrombosis), or insufficient driving blood flow pressure (arterial occlusion), an abnormal waveform pattern will be produced. Waveform pattern recognition algorithms will be used to determine the presence of normal or abnormal waveform patterns indicative of compromised blood flow through the vessel and the probable etiology of the abnormality in blood flow through the vessel. The controller employs a wireless module 310 to output the blood flow waveform pattern to a remote notification device 326 kept by the patient. In addition, an audio jack configured to allow a clinician to listen to the blood flow signal. The wireless module 310 in an example, is any suitable wireless technology transceiver, such as for example any wireless transceiver, designed for short-range, low-power communication and data exchange between fixed and mobile devices. The wireless module 310 receives the blood flow waveform and embeds the blood flow waveform into a wireless signal 328 transmitted to the remote notification device 326.

The remote notification device 326 in an example, is any suitable handheld device configured for using a cellular network including but not limited to smart phones, tablets, and the like. The remote notification device 326 receives the blood flow waveform in the form of the wireless signal 328 and then cellularly transmits the blood flow waveform as a cellular signal 330 to one or more networks of remote server's database 334. The one or more networks of remote server's database 334 receives the blood flow waveform via an upload or any other means, applies the waveform pattern recognition filters and then saves the waveform in a file. In examples, waveform pattern recognition filters may receive the blood flow waveform and apply filters before processing techniques. Applying waveform pattern recognition filters may reduce the processing load required for later processing.

The waveform pattern is stored in the file, which is accessible by the clinician through the clinician notification device 332, in order to allow the clinician to track and examine the waveform pattern for the purpose of remotely monitoring the patient. The clinician notification device 332 in an example, is any suitable handheld device configured for using a cellular network including but not limited to smart phones, tablets, and the like. If pattern recognition algorithms determine a waveform pattern is abnormal, the clinician notification device 332 receives a notification informing the clinician of the abnormal waveform pattern indicative of compromised blood flow through the blood vessels 104 and probable etiology.

In addition, when the one or more networks of remote servers-based pattern recognition algorithm detects the patient blood flow waveform is not within the predetermined pattern range indicating abnormal blood flow through the blood vessels 104, a cellular signal is sent to the remote notification device 326 held by the patient to notify the patient of the abnormal waveform pattern.

The blood flow monitor 100 in an example, comprises an audio jack 324 operatively connected with the signal processor 306 and more particularly with the audio difference amplifier 322 to receive the blood flow signal therefrom. The audio jack 324, which is configured to interface with an earphone, amplified speaker, and the like, provides the blood flow monitor 100 with the capability for a clinician to directly listen to the blood flow signal since the blood flow signal in an example, is an audio signal representative of blood flow through blood vessel 104. The audio jack 324 accordingly permits a clinician to directly monitor the patient because the blood flow signal provides an audible signal representation of blood flow through patient blood vessel 104.

Upon the signal processor 306 generating a blood flow signal, the controller 300 develops a patient heart rate value using the blood flow signal, an illustration of which is shown by the example waveform of FIG. 11. A patient's heart as a flow pump creates a pulsing in blood flowing through the blood vessels 104 such that the velocity of the blood flowing through the blood vessels 104 rhythmically cycles between dip values and peak values as shown in FIG. 11. Illustratively, the velocity of the blood flowing through the blood vessels 104 rhythmically increases from a dip value (D1) to a peak value (P1), decreases from the peak value (P1) to a dip value (D2), and then again increases from the dip value (D2) to a peak value (P2). In accordance with the relationship between patient heart rate and blood flow, two adjacent peak values (P1 and P2 in the example waveform shown in FIG. 11) within the blood flow signal indicate a patient heartbeat whereby the one patient heartbeat represented by P1 and P2 of FIG. 11 divided by a time difference in minutes (T_{P2}−T_{P1}) between the two adjacent peak values (P1 and P2 in the example waveform shown FIG. 11) permits a calculation of a patient heart rate value (HR) using the equation HR=1 (heartbeat)/(T_{P2}−T_{P1} in minutes), which results in a patient heart rate in beats per minute (BPM). In the event (T_{P2}−T_{P1}) is expressed in seconds, HR=60 (heartbeats)/(T_{P2}−T_{P1} in seconds), which produces a patient heart rate in beats per minute (BPM). Although the controller 300 requires location of only two adjacent peak values (P1 and P2 in the example waveform shown in FIG. 11) and the time difference therebetween, which is tracked by the controller 300 during location of P1 and P2, to develop a patient heart rate value, the controller 300 in an example, locates multiple peak values (i.e., more than 2) and then calculates an average of the time differences between adjacent peak values of the multiple peak values in order to develop a patient heart rate value on the basis the accuracy of the blood flow monitor 100 in determining patient heart rate value and thus whether blood is flowing through the blood vessels 104 increases when more than 2 peak values and the time differences between adjacent peak values are used.

The controller 300 in developing a patient heart rate value using the blood flow signal locates a first peak value (P1) within the blood flow signal and a time (T_{P1}) of the first peak value (P1) within the blood flow signal. The controller 300 further locates a second peak value (P2) within the blood flow signal and a time (T_{P2}) of the second peak value (P2) within the blood flow signal. The controller 300 when T_{P1} and T_{P2} are expressed in minutes then calculates a patient heart rate value (HR) in beats per minute using the equation HR=1/(T_{P2}−T_{P1} in minutes). The controller 300 when T_{P1} and T_{P2} are expressed in seconds calculates a patient heart rate value (HR) in beats per minute using the equation HR=60/(T_{P2}−T_{P1} in seconds).

In the alternative, the controller 300 in developing a patient heart rate value using the blood flow signal locates a plurality of peak values within the blood flow signal and a time (T) of each of the plurality of peak values within the blood flow signal. The controller 300 further calculates an average of the time differences between adjacent peak values of the plurality of peak values thereby generating an average time difference (T_{P-P}). The controller 300 when T_{P-P} is expressed in minutes then calculates a patient heart rate value (HR) in beats per minute using the equation HR=1/(T_{P-P} in minutes). The controller 300 when T_{P-P} is expressed in seconds calculates a patient heart rate value (HR) in beats per minute using the equation HR=60/(T_{P-P} in seconds).

When locating peak values within the blood flow signal including a time of the peak values within the blood flow signal, the controller 300 in an example, initiates a search for a dip value on the basis the controller 300 first locates a dip value within the blood flow signal in order to then determine when a peak value occurs within the blood flow signal. The controller 300 after setting a dip value to a maximum value obtains a blood flow sample from the blood flow signal including a time of the blood flow sample within the blood flow signal. The controller 300 in an example, converts the blood flow sample into an energy sample value to produce a positive value energy waveform such as the example energy waveform shown in FIG. 12. When converting the blood flow sample into an energy sample value, the controller 300 further develops a time of the energy sample value within the example energy waveform shown in FIG. 12, which also is the time of the blood flow sample within the blood flow signal on the basis the conversion does not affect the time location of the blood flow sample. The controller 300 compares the energy sample value to the dip value and then sets the energy sample value to the dip value when the comparison determines the energy sample value is less than the dip value. The controller 300 continues to obtain blood flow samples, convert the blood flow samples into energy sample values that form the example energy waveform shown in FIG. 12, develop times of the energy sample values within the example energy waveform shown in FIG. 12, compare a current energy sample value to the dip value, and set the current energy sample value to the dip value until the comparison determines a current energy sample value is greater than the dip value or the comparison determines a predetermined number of energy sample values are less than the dip value.

Upon locating a dip value such as D1 within the example energy waveform shown in FIG. 12, the controller 300 initiates a search for a peak value within the blood flow signal. The controller 300 after setting a peak value to a minimum value obtains a blood flow sample from the blood flow signal including a time of the blood flow sample within the blood flow signal. The controller 300 converts the blood flow sample into an energy sample value to produce the positive value energy waveform illustrated in the example shown in FIG. 12. When converting the blood flow sample into an energy sample value, the controller 300 further develops a time of the energy sample value within the example energy waveform shown in FIG. 12, which also is the time of the blood flow sample within the blood flow signal on the basis the conversion does not affect the time location of the blood flow sample. The controller 300 compares the energy sample value to the peak value and then sets the energy sample value to the peak value when the comparison determines the energy sample value is greater than the peak value. The controller 300 further sets the time of the energy sample value within the example energy waveform shown in FIG. 12 as the time of the peak value. The controller 300 continues to obtain blood flow samples, convert the blood flow samples into energy sample values that form the example energy waveform shown in FIG. 12, develop times of the energy sample values within the example energy waveform shown in FIG. 12, compare a current energy sample value to the peak value, set the current energy sample value to the dip value, and set the time of the current energy sample value as the time of the peak value until the comparison determines a current energy sample value is less than the peak value or the comparison determines a predetermined number of energy sample values are greater than the peak value.

After locating a peak value including a time of the peak value such as a first peak value P1 within the example energy waveform shown in FIG. 12, the controller 300 as previously described locates an adjacent peak value including a time of the adjacent peak value such as a second peak value P2 within the example energy waveform shown in FIG. 12. The controller 300 as previously described then uses the equation HR=1/(T_{P2}−T_{P1} in minutes) or the equation HR=60/(T_{P2}−T_{P1} in seconds) to calculate a patient heart rate value (HR) in beats per minute. In the alternative, the controller 300, when employing multiple peak values and an average of the time differences between adjacent peak values of the multiple peak values to develop a patient heart rate value, locates peak values including a time of the peak values until a desired predetermined number of peak values and times thereof have been acquired. The controller 300 as previously described then uses the equation HR=1/(T_{P-P} in minutes) or the equation HR=60/(T_{P-P} in seconds) to calculate a patient heart rate value (HR) in beats per minute.

With blood flow velocity (v) calculated by the frequency shift experienced by the reference signal when blood is moving through the blood vessels 104 and utilization of a vascular probe with a defined diameter (2.0, 2.5, 3.0 mm), flow volume is calculated by the formula Q=¼ V· π·ø², where Q=flow volume, v=velocity, ø=probe diameter. The total volume of blood flow through the blood vessel 104 between peak values (P1−P2), representing one heart beat, is calculated. The product of flow volume (Q) and a defined conversion factor (C) is cardiac stroke volume (SV) (ml per beat) (SV=Q·C). Stroke volume calculations are performed with every heart beat and averaged over a minute (SVmean). Additionally, stroke volume variance (SVV) is calculated by taking SVmax−SVmin/SVmean. The controller employs a wireless module 310 to output the calculated SV and SVV to a remote notification device 326 kept by the patient. The wireless module 310 receives the SV and SVV calculations and embeds the SV and SVV values into a wireless signal 328 transmitted to the remote notification device 326. The remote notification device 326 receives the SV and SVV values in the form of the wireless signal 328 and then cellularly transmits the SV and SVV values as a cellular signal 330 to a one or more networks of remote servers database 334. The one or more networks of remote servers database 334 receives and saves the SV and SVV values, accessible by the clinician through the clinician notification device 332.

With calculation of the SVmean and patient heart rate (HR), cardiac output (CO) is determined as CO=SVmean×HR. The calculated CO is stored and accessible by the clinician through the clinician notification device 332. In addition, controller 300 may transmit raw or processed measurements or any patient 200 computation to integrated storage device 336 via wired or wireless implementations. Here, integrated storage device 336 is disposed within monitor 100. However, in other examples it may be disposed with patient 200 or in on off-site facility.

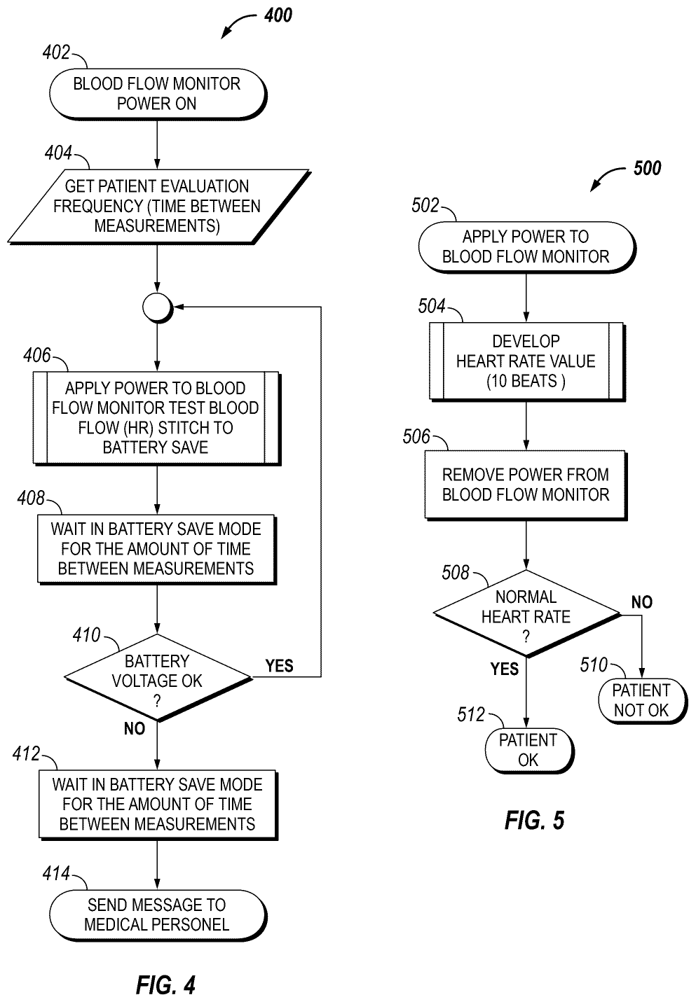

Blood flow monitor 100 using the controller 300 as illustrated in the flow diagrams shown in FIGS. 4-10 operates as follows in monitoring blood flow through the blood vessels 104. Operation of the blood flow monitor 100 in monitoring blood flow through the blood vessels 104 commences with the controller 300 executing workflow 400 including blocks 402-414 as illustrated in FIG. 4. In addition, workflows 500, 600, 700, 800, 900, and 1000 may also be executed via controller 300. However, in other examples workflows 500, 600, 700, 800, 900, and 1000 can be executed via any means and do not require controller 300, even where the workflow describes controller 300, it may still be replaced via any form of processing mechanisms. In block 402 controller 300 may power on of the blood flow monitor 100. Illustratively, a switch associated with the power source 312 is activated to allow supply of power to the controller 300 from the power source 312 and further a distribution of power throughout the blood flow monitor 100 under the control of the controller 300.

In block 404, patient evaluation frequency may be obtained. In examples, measurements from probe 106 (e.g., referring to FIG. 1) may be transmitted to monitor 100. In examples, a patient evaluation frequency may also be referred to as a sample frequency rate. As discussed above, monitor 100 may read a patient evaluation frequency which is a predetermined time period the controller 300 waits between developing a blood flow signal utilized in developing a blood flow waveform as well as systemic hemodynamic or systemic blood flow data. In examples, the time period may be preset or adjusted by a clinician or any other operator. The controller 300 intermittently develops the blood flow signal and systemic hemodynamic or systemic blood flow data to limit the amount of time the power source 312 supplies power throughout the blood flow monitor 100. The power source 312 when the controller 300 is in wait mode supplies only the power necessary for the controller 300 to determine expiration of the predetermined time period and therefore when to generate another blood flow signal. The controller 300 in intermittently developing a blood flow signal provides the blood flow monitor 100 with a power saving mode that allows the power source 312 to be a portable power source, such as, for example, a battery, whereby the blood flow monitor 100 is wearable by the patient and thus portable. As an illustration which is not to be considered limiting, a patient normally is monitored using the blood flow monitor 100 for up to 5 days, although the patient monitoring period may be extended as needed, with the patient evaluation frequency being once every 1 minute up to once every 4 minutes, although the patient evaluation frequency may be lessened or extended under circumstances where less frequent monitoring may safely occur. One of ordinary skill in the art will recognize the physical size of the power source 312 may be selected to permit operation of the blood flow monitor 100 for the entire patient monitoring time period. Alternatively, a reduction in the size of the blood flow monitor 100 may be achieved through a reduction in the physical size of the power source 312 with the power source 312 accordingly requiring charging or replacement during the patient monitoring time period.

In block 406, development of a blood flow signal utilized in developing a blood flow wave may be formed to determine if the patient is experiencing abnormal blood flow through the blood vessels 104. The controller 300 in developing the blood flow signal performs block 406 by calling and then executing workflow 500 including blocks 502-512 as illustrated in FIG. 5. In block 502, controller 300 may control power source 312 to apply a voltage thus supplying power to the blood flow monitor 100 and more particularly to the reference signal generator 302 (oscillator 314 and amplifier 316), the transmitter/receiver 304, the signal processor 306 (doppler receiver amplifier 318, doppler mixer detector 320, and audio difference amplifier 322), the alarm 308, and the wireless module 310. With the blood flow monitor 100 powered, the reference signal generator 302 generates the reference signal for use by the transmitter/receiver 304 as well as the signal processor 306 and more particularly the doppler mixer detector 320 of the signal processor 306. In addition, the signal processor 306 is ready to receive doppler shifted signal values from the transmitter/receiver 304 and then process the doppler shifted signal values into blood flow signal values that form a blood flow signal. Upon the powering of the blood flow monitor 100, the controller 300 proceeds to block 504 and continues development of the blood flow signal utilized in determining if the patient is experiencing an abnormal blood flow through the blood vessels 104.

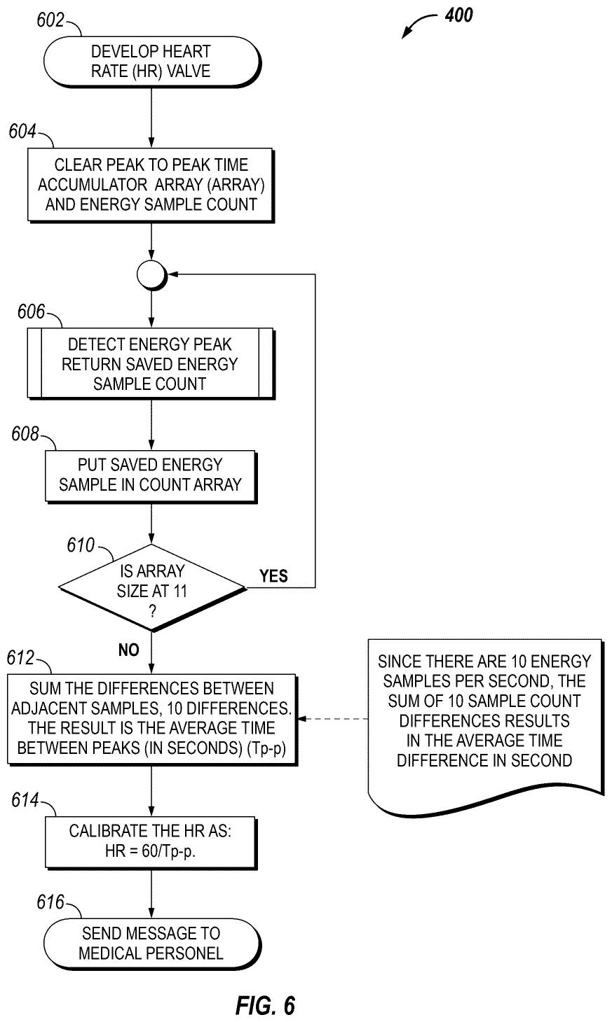

Additionally, the controller 300 in order to determine the patient's heart rate performs block 504 by calling and then executing workflow 600 including blocks 602-616 as illustrated in FIG. 6. In block 602, controller 300 may initiate development of the heart rate value, which comprises a detection of peak values in terms of energy and locations thereof within the blood flow signal an example of which is shown in FIG. 11. In block 604, a peak-to-peak time accumulator array and an energy sample count may be cleared. The peak to peak time accumulator array as will be described more fully herein tracks the number of energy peaks detected within the blood flow signal. The energy sample count as will be described more fully herein provides a count of the number of energy sample values taken from the blood flow signal during development of the energy peaks. Moreover, as will be described more fully herein, the energy sample count facilitates timing determinations utilized in establishing energy peak locations. Upon clearing the peak to peak time accumulator array and the energy sample count, in block 606 detection of an energy peak and a location thereof utilized in developing the heart rate value may be commended. The detection of an energy peak and a location thereof will be described herein, including with reference to FIG. 12 which illustrates peaks in energy and locations thereof and dips in energy and locations thereof within an example energy waveform developed from the energy sample values taken from the blood flow signal.

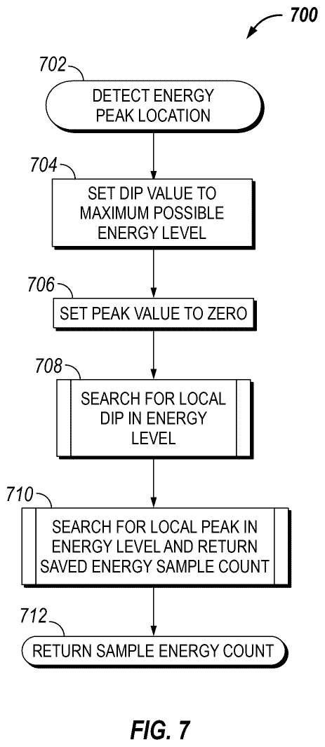

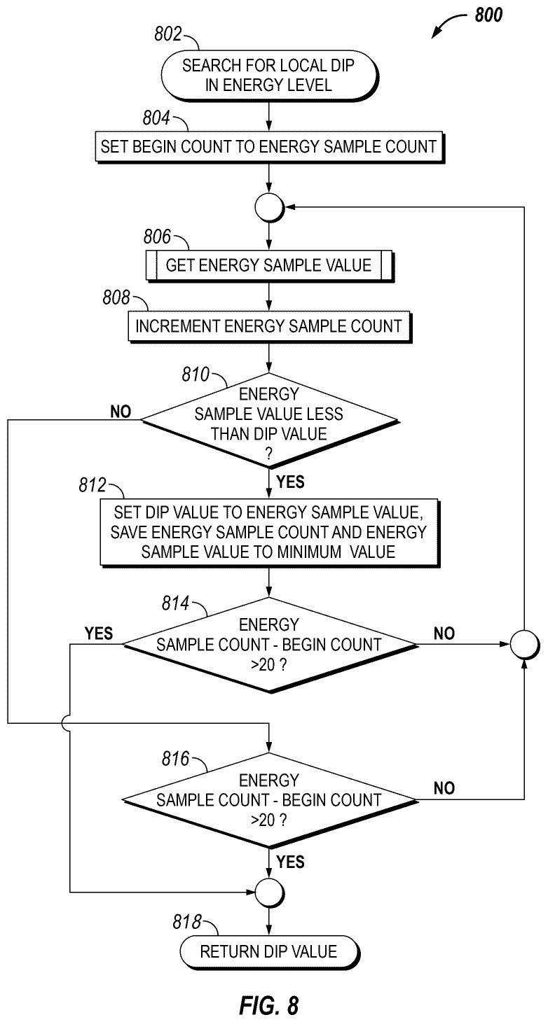

The controller 300 in detecting the energy peak and location thereof performs block 606 by calling and then executing workflow 700 including blocks 702-712 as illustrated in FIG. 7 The In block 702 where the controller 300 initiates detection of an energy peak and a location thereof. In block 704 a dip value to a maximum possible energy value may be set. In examples this may be an energy value predetermined to be greater than any energy value detected by the blood flow monitor 100 during a monitoring of blood flow through the blood vessels 104 of the patient 200. Upon setting an initial dip value in block 704, which is an initial dip value, the controller in block 706 sets a peak value to 0. With the initial peak value set in block 706, which is an initial peak value, the controller 300 proceeds to block 708 and continues detection of an energy peak and a location thereof by calling and then executing workflow 800 including blocks 802-818 as illustrated in FIG. 8.

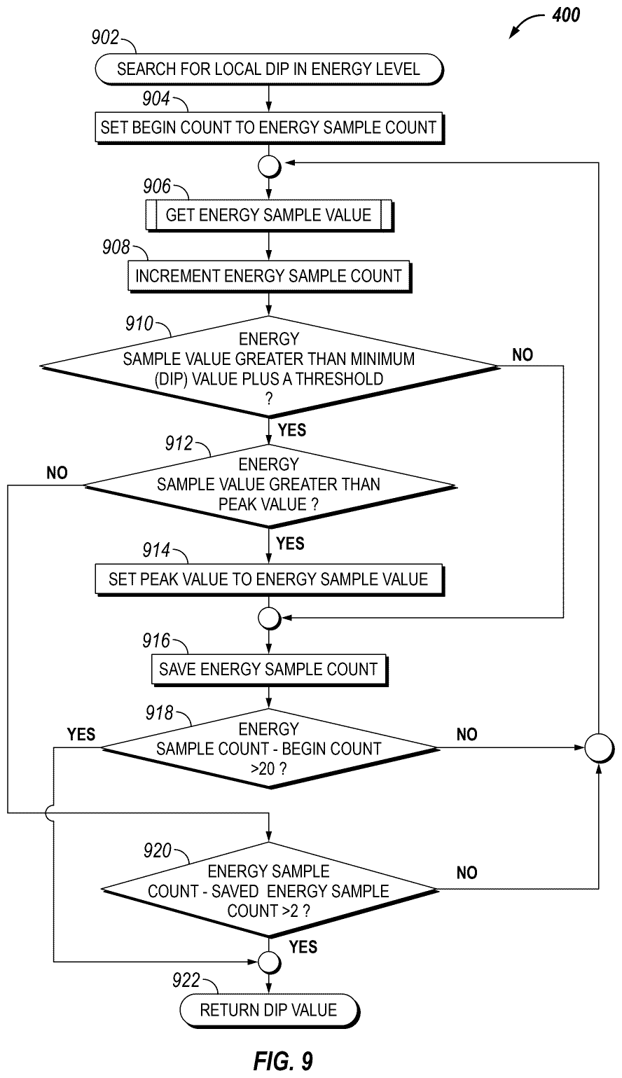

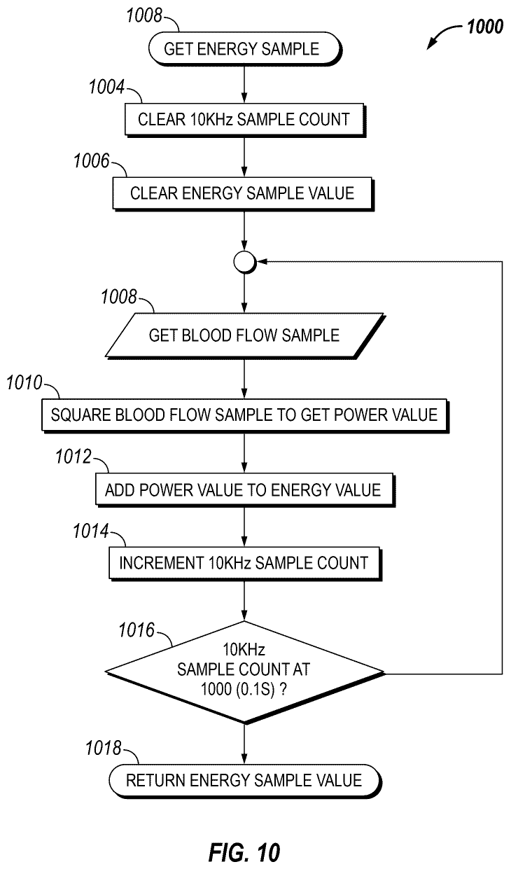

In block 802 controller 300 may initiate a search for a local dip in energy level on the basis the controller 300 first locates an energy dip within the blood flow signal in order to then determine when an energy peak occurs within the blood flow signal during execution of workflow 100 including blocks 902-922 as illustrated in FIG. 9. In block 804 a begin count to the energy sample count may be set in order to allow a timing determination that establishes a time limit for finding an energy dip within the blood flow signal. After setting the begin count, controller 300 may proceed to block 806 and detection of an energy dip by calling and then executing workflow 1000 including blocks 1002-1018 as illustrated in FIG. 10 may be continued.

Workflow 1000 may proceed. In block 1002 where the controller 300 commences generation of an energy sample value developed from the blood flow signal. The controller 300 in block 1004 clears a sample count, a 10 KHz sample count in an example, which is utilized in tracking a predetermined number of blood flow samples developed from the blood flow signal. The predetermined number of blood flow samples, in examples 1,000, may be used in generating the energy sample value. Moreover, the 10 KHz sample count based upon the sample count being associated with 100,000 counts per a time period of 1 second provides a time count of 0.1 of a second (1,000 samples over 100,000 seconds) for use in timing determinations employed to establish energy peak locations. In other examples, the sample count may be 20 KHz. The controller 300 in an example utilizes one thousand 1,000 blood flow samples taken over the 0.1 of a second time frame within the blood flow signal because the accuracy of the blood flow monitor 100 in determining patient heart rate and thus whether blood is flowing through the blood vessels 104 increases when multiple blood flow samples are used. Nevertheless, one of ordinary skill in the art will recognize that a different predetermined number of blood flow samples, including 1 or more, may be used by the controller 300 in generating an energy sample value. In addition, one of ordinary skill in the art will recognize that different sample counts other than the 10 KHz sample count producing the 0.1 of a second time frame may be used. After clearing the 10 KHz sample count, the controller 300 in block 1006 clears the energy sample value to permit development of another energy sample value.

Upon clearing the energy sample value, in block 1008 development of a blood flow sample from the blood flow signal, such as the example waveform of the blood flow signal illustrated in FIG. 11, which is produced through the intermittent control of the transmitter/receiver 304 by the controller 300 as previously described may be applied. In examples, controller 300 accesses the signal processor 306 and more particularly the audio difference amplifier 322 and samples the blood flow signal generated by the signal processor 306 in order to obtain the blood flow sample, which, in an example, is the current voltage value of the blood flow signal at the sample point within the blood flow signal. The controller 300 prior to inputting the blood flow sample uses the audio difference amplifier 322 of the signal processor 306 to amplify the blood flow sample taken from the blood flow signal to a signal level suitable for digitizing and subsequent use by the controller 300 in generating the energy sample value.

Once blood flow sample is obtained, including digitizing the blood flow sample, in block 1010 generating a power value from the blood flow sample by squaring the blood flow sample on the basis a power value is proportional to a square of a voltage value may be performed. Moreover, the controller 300 squares the blood flow sample to ensure the power value is a positive number. In block 1012 through adding the power value to a current value of the energy sample value, which begins at 0 due to execution of block 1006, in order to produce the energy sample value as single value that, in an example, is a representation of the energy within the blood flow signal over 0.1 of a second. After adding the power value to generate the energy sample value, in block 1014 increments the 10 KHz sample count may be applied. In block 1016, it may be determined if the 10 KHz sample count is 1,000 indicating the 0.1 of a second time frame within the blood flow signal has been reached. When the 10 KHz sample count is less than 1,000. In block 1008 and obtains another blood flow sample as previously described. Controller 300 in block 1010 generates a power value from the blood flow sample and then in block 1012 adds the power value to the current value of the energy sample value to produce the energy sample value as a single value including the power value. The controller 300 again in block 1014 increments the 10 KHz sample count and then in block 1016 determines if the 10 KHz sample count is 1,000. The controller 300 continues to add power values to the energy sample value until the 10 KHz sample count is 1,000 indicating the energy sample value has reached the representation of the energy within the blood flow signal over 0.1 of a second. The controller 300 when the 10 KHz sample count is 1,000 proceeds to block 1018 and returns the energy sample value to the calling workflow, which is one of the workflow 800 as illustrated in FIG. 8 or the workflow 100 as illustrated in FIG. 9. Nevertheless, the controller 300 at this point returns to workflow 800 at block 806 thereof on the basis the controller 300 is locating an energy dip within the blood flow signal.

The controller 300 proceeds from block 806 and executes block 808 by incrementing the energy sample count in order to provide an increasing number count utilized in tracking each energy sample value taken from the blood flow signal regardless of whether the energy sample value is returned for use in locating a dip or a peak. Moreover, the energy sample count facilitates timing determinations utilized in establishing energy peak locations on the basis each energy sample value returned from the workflow 1000 is generated in 0.1 of a second such that the number count of the energy sample count associated with the returned energy sample value multiplied by 0.1 of a second produces the time frame of the returned energy sample value within the example energy waveform shown in FIG. 12. As an illustration, the first energy sample value has a time frame of 0.1 of a second within the example energy waveform, whereas a third energy sample value has a time frame of 0.3 of a second within the example energy waveform.

The controller in block 810 determines whether the energy sample value returned from the workflow 1000 is less than the dip value, which in block 704 was initially set to a maximum possible energy value whereby the first energy sample value returned to the workflow 800 is less than the dip value. In accordance therewith, the controller 300 initially proceeds from block 810 to block 812 and sets the dip value to the energy sample value, saves the energy sample count associated with the energy sample value as a saved energy sample count associated with the dip value, and sets the energy sample value, which is the dip value, to a minimum value. The controller 300 in block 814 subtracts the begin count from the energy sample count of block 808 to ensure an energy dip within the blood flow signal is located within a predetermined number of energy sample values, which in an example, is 20 thereby establishing a time limit of 2 seconds (20 energy sample values multiplied by 0.1 of a second equals 2 seconds).

As long as the 2 second time limit has not been exceeded, the controller exits block 814 and returns to block 806 where the controller 300 calls and then executes workflow 1000 to obtain another energy sample value as previously described. After returning another energy sample value to workflow 800, the controller 300 in block 808 increments the energy sample count and then in block 810 determines whether the energy sample value returned from the workflow 1000 is less than the dip value. If the returned energy sample value is less than the dip value, as shown by S2 relative to S1 in FIG. 12, the controller 300 proceeds from block 810 to block 812 and sets the dip value to the energy sample value, saves the energy sample count associated with the energy sample value as a saved energy sample count associated with the dip value, and sets the energy sample value, which is the dip value, to a minimum value.

The controller 300 continues to obtain energy sample values that form the example energy waveform shown in FIG. 12, increment the energy sample count, determine whether returned energy sample values are less than the dip value, and set the dip value to the energy sample value and save the energy sample count as a saved energy sample count associated with the dip value until the controller in block 810 determines a returned energy sample value is not less than the dip value indicating the returned energy sample values are increasing or the controller 300 in block 814 determines the 2 second time limit has been exceeded on the basis the controller 300 in blocks 810 and 812 decreasing energy sample values. The controller 300 accordingly executes block 814 in order to ensure an energy dip such as D1 shown in FIG. 12 is located within 20 energy sample values or 2 seconds because a failure to locate an energy dip within 2 seconds likely indicates abnormal blood flow through blood vessels 104, although confirmation of this occurs during execution of workflow 100. The controller 300 when the 2 second time limit is exceeded proceeds to block 818 and then returns to the calling workflow 700 in block 708 as illustrated in FIG. 7.

When the controller in block 810 determines a returned energy sample value is not less than the dip value, the controller proceeds to block 816 and subtracts the saved energy sample count, which is the energy sample count associated with the dip value, from the energy sample count associated with the returned energy sample value in order to provide a verification returned energy samples are increasing. The controller 300 in block 816 subtracts the saved energy sample count from the energy sample count to determine whether returned energy samples remain increasing within a predetermined number of energy sample values, which in an example, is 2 thereby establishing a time limit of 0.2 seconds (2 energy sample values multiplied by 0.1 of a second equals 0.2 seconds). As long as the 0.2 second time limit has not been exceeded, the controller exits block 816 and returns to block 806 where the controller 300 calls and then executes workflow 1000 to obtain another energy sample value as previously described. After returning another energy sample value to workflow 800, the controller 300 in block 808 increments the energy sample count and then in block 810 determines whether the energy sample value returned from the workflow 1000 is less than the dip value. If the returned energy sample value is not less than the dip value, the controller 300 proceeds from block 810 to block 816 and checks if the 0.2 time limit has been exceeded. The controller 300 when the 0.2 second time limit is exceeded proceeds to block 818 and then returns to the calling workflow 700 in block 708 as illustrated in FIG. 7.

The controller 300 proceeds from block 708 and executes block 710 to commence searching for an energy peak within the blood flow signal on the basis an energy peak now may located since an energy dip within the blood flow signal such as D1 shown in FIG. 12 has been established. The controller 300 in executing block 710 commences detection of an energy peak and a location thereof by calling and then executing workflow 100 including blocks 8002-920 as illustrated in FIG. 9.

The controller 300 begins workflow 100 in block 902 where the controller 300 initiates a search for a local peak in energy level within the blood flow signal. The controller 300 in block 904 sets a begin count to the energy sample count in order to allow a timing determination that establishes a time limit for finding an energy peak within the blood flow signal. After setting the begin count, the controller 300 proceeds to block 906 and continues detection of an energy peak by calling and then executing workflow 1000 including blocks 1002-1018 as illustrated in FIG. 10 in order to obtain an energy sample value as previously described. The controller 300 after employing workflow 1000 to develop an energy sample value as previously described returns the energy sample value to workflow 100 at block 906 thereof on the basis the controller 300 is locating an energy peak within the blood flow signal.

The controller 300 proceeds from block 906 and executes block 908 by incrementing the energy sample count in order to provide an increasing number count utilized in tracking each energy sample value taken from the blood flow signal regardless of whether the energy sample value is returned for use in locating a dip or a peak. Moreover, the energy sample count facilitates timing determinations utilized in establishing energy peak locations on the basis each energy sample value returned from the workflow 1000 is generated in 0.1 of a second such that the number count of the energy sample count associated with the returned energy sample value multiplied by 0.1 of a second produces the time frame of the returned energy sample value within the example energy waveform shown in FIG. 12. As an illustration, a sixth energy sample value has a time frame of 0.6 of a second within the example energy waveform, whereas an eleventh energy sample value has a time frame of 1.1 seconds within the example energy waveform.

The controller in block 910 determines whether the energy sample value returned from the workflow 1000 is greater than a minimum value, which in an example, is the dip value such as D1 shown in FIG. 12 established by the controller 300 during execution of workflow 800 as previously described, plus a threshold, which in an example, is a minimal energy value comprised with the minimum value to ensure the energy sample value returned from the workflow 1000 is not noise at the minimum value indicating abnormal blood flow through the blood vessels 104. When the controller in block 910 determines the returned energy sample value is not greater than the minimum value plus the threshold indicating abnormal blood flow through blood vessels 104, the controller 300 exits block 910, proceeds to block 916 where the controller 300 saves the energy sample count associated with the energy sample value as a saved energy sample count, and then executes block 918 as will be described more fully herein. Alternatively, when the controller in block 910 determines the returned energy sample value is greater than the minimum value plus the threshold, the controller 300 exits block 910 and executes block 912.

The controller in block 912 determines whether the energy sample value returned from the workflow 1000 is greater than the peak value, which in block 706 was initially set to 0 whereby the first energy sample value returned to workflow 100 is greater than the peak value. In accordance therewith, the controller 300 initially proceeds from block 912 to block 914 and sets the peak value to the energy sample value. The controller 300 further proceeds from block 914 to block 916 and saves the energy sample count associated with the energy sample value as a saved energy sample count associated with the peak value. The controller 300 in block 918 subtracts the begin count from the energy sample count of block 908 to ensure an energy peak within the blood flow signal is located within a predetermined number of energy sample values, which in an example, is 300 thereby establishing a time limit of 2 seconds (20 energy sample values multiplied by 0.1 of a second equals 2 seconds).

As long as the 2 second time limit has not been exceeded, the controller exits block 918 and returns to block 906 where the controller 300 calls and then executes workflow 1000 to obtain another energy sample value as previously described. After returning another energy sample value to workflow 100, the controller 300 in block 908 increments the energy sample count and then in block 910 determines whether the energy sample value returned from the workflow 1000 is greater than the minimum value plus the threshold. The controller 300 while the returned energy sample value remains greater than the minimum value plus the threshold proceeds to block 912 and determines whether the energy sample value returned from the workflow 1000 is greater than the peak value. If the returned energy sample value is greater, as shown by S4 relative to S3 in FIG. 12, the controller 300 proceeds from block 912 to block 914 and sets the peak value to the energy sample value. The controller 300 further proceeds from block 914 to block 916 and saves the energy sample count associated with the energy sample value as a saved energy sample count associated with the peak value.

The controller 300 continues to obtain energy sample values that form the example energy waveform shown in FIG. 12, increment the energy sample count, determine whether returned energy sample values are greater than the minimum value plus the threshold and the peak value, set the peak value to the energy sample value, and save the energy sample count as a saved energy sample count associated with the peak value until the controller 300 in block 912 determines a returned energy sample value is not greater than the peak value indicating the returned energy sample values are decreasing or the controller 300 in block 918 determines the 2 second time limit has been exceeded on the basis the controller 300 in blocks 912, 914, and 916 increasing energy sample values. The controller 300 accordingly executes block 918 in order to ensure an energy peak such as P1 shown in FIG. 12 is located within 20 energy sample values or 2 seconds because a failure to locate an energy peak within 2 seconds indicates abnormal blood flow through the blood vessels 104. The controller 300 when the 2 second time limit is exceeded proceeds to block 922 and then returns to the calling workflow 700 in block 710 as illustrated in FIG. 7.

When the controller in block 912 determines a returned energy sample value is not greater than the peak value, the controller 300 proceeds to block 920 and subtracts the saved energy sample count, which is the energy sample count associated with the peak value, from the energy sample count associated with the returned energy sample value in order to provide a verification returned energy samples are decreasing. The controller 300 in block 920 subtracts the saved energy sample count from the energy sample count to determine whether returned energy samples remain decreasing within a predetermined number of energy sample values, which in an example, is 2 thereby establishing a time limit of 0.2 seconds (2 energy sample values multiplied by 0.1 of a second equals 0.2 seconds). As long as the 0.2 second time limit has not been exceeded, the controller exits block 920 and returns to block 906 where the controller 300 calls and then executes workflow 1000 to obtain another energy sample value as previously described. After returning another energy sample value to workflow 100, the controller 300 in block 908 increments the energy sample count and then in block 910 determines whether the energy sample value returned from the workflow 1000 is greater than the minimum value plus the threshold. The controller 300 when the returned energy sample value is greater than the minimum value plus the threshold proceeds to block 912 and determines whether the returned energy sample value is greater than the peak value. If the returned energy sample value is not greater than the peak value, the controller 300 proceeds from block 910 to block 920 and checks if the 0.2 time limit has been exceeded. The controller 300 once the 0.2 second time limit is exceeded proceeds to block 922 and then returns to the calling workflow 700 in block 710 as illustrated in FIG. 7.

Returning to block 910 and the decision of whether a returned energy sample value is greater than the minimum value plus the threshold, the controller 300 upon establishing a returned energy sample value is not greater than the minimum value plus the threshold proceeds to block 918 for a determination of whether 300 returned energy sample values are less than the minimum value plus the threshold such that the 2 second time limit is exceeded. As long as the 2 second time limit has not been exceeded, the controller exits block 918 and returns to block 906 where the controller 300 calls and then executes workflow 1000 to obtain another energy sample value as previously described. The controller 300 continues to obtain energy sample values, increment the energy sample count, determine whether returned energy sample values are greater than the minimum value plus the threshold and the peak value, save the energy sample count associated with the energy sample value as a saved energy sample count, and execute block 918 until the controller 300 in block 918 determines the 2 second time limit has been exceeded on the basis the controller 300 energy sample values less than the minimum value plus the threshold. The controller 300 accordingly executes block 918 in order to ensure an energy peak such as P1 shown in FIG. 12 is located within 300 energy sample values or 2 seconds because a failure to locate an energy peak within 2 seconds indicates abnormal blood flow through the blood vessels 104. The controller 5300 when the 2 second time limit is exceeded proceeds to block 922 and then returns to the calling workflow 700 in block 710 as illustrated in FIG. 7.

The controller 300 upon returning to block 710 continues execution thereof by establishing the location of the energy peak within the example energy waveform shown in FIG. 12, such as energy peak P1, which was found during execution of workflow 100 as previously described, using the saved energy sample count. More particularly, the controller 300 when executing workflow 100 determines the peak value, which is the final increasing energy sample value found after a dip value but prior to a subsequent decreasing energy sample value. Moreover, the controller 300 upon determining an energy peak within the example energy waveform establishes the location of the peak value within the example energy waveform shown in FIG. 12 through saving the energy sample count associated with the energy sample value determined to be the peak value as a saved energy sample count associated with the peak value. The saved energy sample count associated with the peak value establishes the energy peak location on the basis each energy sample value returned from the workflow 1000 is generated in 0.1 of a second such that the energy sample count associated with the returned energy sample value multiplied by 0.1 of a second produces the time frame of the returned energy sample value within the example energy waveform shown in FIG. 12. As an illustration, if a sixth energy sample value, which has an energy sample count of 6, is determined to be the peak value, then the peak value would comprise a time frame location of 0.6 of a second within the example energy waveform due to a saved energy sample count of 6 being multiplied by 0.1 of a second. After using the saved energy sample count associated with the peak value to establish the location of the energy peak within the example energy waveform shown in FIG. 12, the controller 300 proceeds to block 712 and then returns to the calling workflow 600 in block 606 as illustrated in FIG. 6.

The controller 300 upon returning to block 606 continues execution thereof by returning the saved energy sample count associated with the peak value for use in developing a heart rate value of a patient. As an illustration, the controller in block 606 returns from workflows 700, 800, 100, and 1000 as previously described a saved energy sample count associated with the first peak P1 shown in FIG. 12, which illustratively may be a sixth energy sample value having an energy sample count of 6 such that the time frame location of P1 is 0.6 of a second within the example energy waveform. The controller 300 in block 608 places the saved energy sample count associated with the peak value returned from workflows 700, 800, 100, and 1000 into the peak to peak time accumulator array in order to hold the saved energy sample count for use in developing a heart rate value of a patient. The controller 300 in block 610 determines whether the peak to peak time accumulator array comprises 11 saved energy sample counts associated with 11 peak values. When the peak to peak time accumulator array comprises 11 saved energy sample counts associated with 11 peak values, the controller 300 proceeds to block 612 and, as will be described more fully herein, employs the 11 saved energy sample counts in developing a patient heart rate value. Alternatively, when the peak to peak time accumulator array comprises less than 11 saved energy sample counts associated with 11 peak values, the controller 300 proceeds to block 606 in order to detect next energy peak and a location thereof within the example energy waveform shown in FIG. 12 through executing workflows 700, 800, 100, and 1000 as previously described.

The controller 300 upon executing workflows 700, 800, 100, and 1000 as previously described returns a next saved energy sample count associated with a next peak value for use in developing a patient heart rate value. As an illustration, the controller in block 606 returns from workflows 700, 800, 100, and 1000 as previously described a saved energy sample count associated with the second peak P2 shown in FIG. 12, which illustratively may be a fourteenth energy sample value having an energy sample count of 14 such that the time frame location of P2 is 1.4 seconds within the example energy waveform. The controller 300 in block 608 places the next saved energy sample count associated with the next peak value returned from workflows 700, 800, 100, and 1000 into the peak to peak time accumulator array in order to hold the next saved energy sample count for use in developing a patient heart rate value. The controller 300 continues executing workflows 700, 800, 100, and 1000 as previously described to return saved energy sample counts associated with peak values for use in developing a patient heart rate value until the controller 300 in block 610 determines the peak to peak time accumulator array comprises 11 saved energy sample counts associated with 11 peak values.

Upon determining the peak to peak time accumulator array comprises 11 saved energy sample counts associated with 11 peak values, the controller 300 proceeds to block 612 and executes block 612 to commence development of a patient heart rate value. In accordance with the relationship between patient heart rate and blood flow, adjacent peak values within the example energy waveform shown in FIG. 12 located by the controller 300 during execution of workflows 700, 800, 100, and 1000 indicates a patient heartbeat whereby a time difference between at least two adjacent peaks permits a calculation of a patient heart rate value as will be described more fully herein. Illustratively, a time of P2 minus a time of P1 (T_{P2-P1}), such as, for example, 1.4 seconds (T_{P2})−0.6 seconds (T_{P1})=0.8 seconds, permits a calculation of a patient heart rate value. While the controller 300 requires location of only 2 adjacent peak values, which represent one patient heartbeat, and the time difference between the 2 adjacent peak values to develop a patient heart rate value, the controller 300 in an example, locates 11 peak values (i.e., 10 heartbeats) and then calculates an average of the 10 time differences between adjacent peak values of the 11 peak values in order to develop a patient heart rate value on the basis the accuracy of the blood flow monitor 100 in determining patient heart rate value and thus whether blood is flowing through the blood vessels 104 increases when more than 2 peak values and the time differences between adjacent peak values are used. The controller 300 accordingly in executing block 612 sums the time difference between each adjacent saved energy sample count of the 11 saved energy sample counts and then calculates the average of the time differences between the 11 peak values (i.e., 10 heartbeats) to generate an average time between peak values in seconds (T_{P-P}), which, illustratively, may equal 0.7 seconds. In the event the controller 300 in workflow 100 determines energy sample values are not greater than the minimum value plus the threshold or an energy peak is not located within the 2 second time limit, the controller 300 in blocks 606-610 establishes the saved energy sample counts as being 40 counts or 4 seconds apart on the basis block 918 in workflow 100 utilizes 20 energy sample values. In accordance therewith, the controller 300 in executing block 612 generates an average time between peak values in seconds (T_{P-P}) of 4 seconds, which, as will be described more fully herein.

After generating an average time between peak values in seconds (T_{P-P}) in block 612, the controller 300 proceeds to block 614 and calculates patient heart rate value (HR) as HR=(60 seconds) divided by T_{P-P} (the average time between heartbeats in seconds) or HR=60/T_{P-P}. As an illustration, with T_{P-P} equaling 0.7 seconds, HR=60/0.7 or a heart rate value of 85.7 beats per minute. In the example of calculating a heart rate value using the 2 peak values of P1 and P2 with T_{P2-P1} equaling 0.8 seconds, HR=60/0.8 or a heart rate value of 75 beats per minute. In the event T_{P-P} is determined in minutes, the patient heart rate value is calculated using HR=1/(T_{P-P} in minutes).

In the alternative, the controller 300 in blocks 612 and 614 may use the number value of the saved energy sample count associated with the peak value. As an illustration, if the first peak P1 shown in FIG. 12 is a sixth energy sample value having an energy sample count of 6 and the second peak P2 shown in FIG. 12 is a fourteenth energy sample value having an energy sample count of 14, T_{P2-P1} would equal 8. In accordance with using the number value of the saved energy sample count, the controller 300 in executing block 612 to generate an average time between peak values would return a T_{P-P} of 7 instead of 0.7 seconds. HR accordingly would be calculated as HR=600/T_{P-P} in order to account for the number value of the saved energy sample count instead of the 0.1 of a second time value. As an illustration, with T_{P-P} equaling a number value of 7, HR=600/7 or a heart rate value of 85.7 beats per minute.

The controller 300 upon returning to block 504 continues execution thereof by returning the calculated patient heart value (HR) to workflow 500, such as, for example, a heart rate value of 85.7 beats per minute. The controller 300 then proceeds to block 506 and removes power from the blood flow monitor 100 in order to initiate the power saving mode that allows the power source 312 to be a portable power source, such as, for example, a battery. Then in block 508 it is determined of the heart rate is normal. If it us workflow 500 proceeds to block 512, if it is not it proceeds to block 510.

The controller 300 after returning to the calling workflow 400 in block 406 continues execution of block 406 by switching to the power saving mode of the blood flow monitor 100 whereby the controller 300 stops the power source 312 from supplying power throughout the blood flow monitor 100. The controller 300 exits block 406 and proceeds to block 408 where the controller 300 waits until expiration of the predetermined time period comprising the patient evaluation frequency. Upon expiration of the predetermined time period comprising the patient evaluation frequency, the controller 300 proceeds to block 410 and compares a voltage level output of the power source 312 with a minimum voltage level. While the minimum voltage level may be a preset voltage level of the blood flow monitor 100, in an example, the blood flow monitor 100 comprises an input feature useable by a clinician to enter the minimum voltage level.

When the controller 300 in block 410 determines the voltage level output of the power source 312 is greater than the minimum voltage level, the controller 300 returns to block 406 and as previously described commences development of another heart rate value utilized in determining if the patient is experiencing abnormal blood flow through the blood vessels 104. Alternatively, when the controller 300 in block 410 determines the voltage level output of the power source 312 is less than or equal to the minimum voltage level, the controller 300 proceeds 412 where the controller 300 waits until expiration of the predetermined time period comprising the patient evaluation frequency. Upon expiration of the predetermined time period comprising the patient evaluation frequency, the controller 300 proceeds to block 414 and employs the wireless module 310 to output to the remote notification device 326 a power source status signal indicating the voltage level output of the power source 312 is less than or equal to the minimum voltage level. As an illustration, the power source status signal in an example, blood flow monitor 100 comprises a data signal including text messaging for the clinician informing the clinician the power source requires replacement. The wireless module 310 receives the power source status signal and embeds the power source status signal into the wireless signal 328 transmitted to the remote notification device 326. The remote notification device 326 receives the power source status signal in the form of the wireless signal 328 and then cellularly transmits the power source status signal as a cellular signal 330 to the clinician notification device 332. The clinician notification device 332 receives the power source status signal in the form of the cellular signal 330 in order to deliver the power source status signal to the clinician. As an illustration, the clinician notification device 332 receives the power source status signal as a text message informing the clinician the power source 312 requires replacement. During replacement of the power source 312, the blood flow monitor is powered off such that, after replacement of the power source 312 and a powering on of the blood flow monitor 100, the controller 300 commences workflow 400 in block 402 as previously described.

Although the present invention has been described in terms of the foregoing examples, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

Throughout the patient monitoring period, the controller 300 employs the wireless module 310 to output a blood flow signal to a remote notification device 326 kept by the patient. The remote notification device 326 in an example, is any suitable handheld device configured for using a cellular network including but not limited to smart phones, tablets, and the like. The remote notification device 326 receives the blood flow status signal in the form of the wireless signal 328. Herein, flow status may refer to volume or rate of blood moving to, through, or out of a free tissue flap. Upon receiving the blood flow signal, a blood flow waveform may be generated by the remote notification device 326. In examples, the blood flow waveform may be generated on monitor 100 or on one or more networks of remote servers database 334. In addition, through cellular communication with the one or more networks of remote servers database 334, the blood flow waveform will be stored and relayed to the clinician notification device 332. The clinician notification device 332 in an example, is any suitable handheld device configured for using a cellular network including but not limited to smart phones, tablets, and the like. Concurrent application of filters by the remote notification device 326 will be used to determine whether the wave form pattern generated is consistent with normal or abnormal blood flow through blood vessel 104. In addition, filters may also be applied on one or more networks of remote servers database 334. If considered an abnormal blood flow pattern, the remote notification device 326 will notify the patient through an audible alert. Similarly, through cellular communication with the one or more networks of remote servers database 334, the abnormal blood flow alert will be relayed to the clinician notification device 332 through an audible alert or text message.

The controller 300 upon generating the patient heart rate (HR), stroke volume (SV), stroke volume variability (SVV), and cardiac output (CO), employs the wireless module 310 to output the calculated hemodynamic measurements to the remote notification device 326 kept by the patient. The remote notification device 326 cellularly transmits calculated measurements to the clinician notification device 332 and one or more networks of remote servers database 334 for storage. Similarly, location of the generation of hemodynamic measurements may be determined.

The systems and methods may include any of the various features disclosed herein, including one or more of the following statements.

Statement 1. A method comprising: receiving a doppler shifted signal from a probe installed at least partially in a free tissue flap; storing the doppler shifted signal in an integrated storage device or uploading the doppler shifted signal in a one or more networks of remote servers; processing at least part of the doppler shifted signal on the integrated storage device or on the one or more networks of remote servers with a signal processor to form a blood flow sample of the free tissue flap; and determining a qualitative blood flow status or a quantitative blood flow status of the free tissue flap with at least one blood flow sample.

Statement 2. The method of claim 1, further comprising defining a sample frequency rate, wherein the sample frequency is predetermined or manually adjusted by a clinician or any other operator.

Statement 3. The method of claim 1, further comprising applying power to a blood flow monitor.

Statement 4. The method of claim 1, wherein the doppler shifted signal is returned to a transmitter/receiver.

Statement 5. The method of claim 1, wherein the probe is any form of a doppler shifted probe, any form of a vascular probe, and/or a cook probe.

Statement 6. The method of claim 1, applying a waveform pattern recognition filter to a generated waveform produced from the doppler shifted signal and saving the waveform in a file on a integrated storage device or the one or more networks of remote servers.

Statement 7. The method of claim 1, wherein the signal processor: receives the doppler shifted signal with a doppler receiver amplifier; amplifies the doppler shifted signal with the doppler receiver amplifier; multiplies a reference signal received from a reference signal generator with the doppler shifted signal to form a blood flow signal; and samples the blood flow signal to obtain a blood flow sample.

Statement 8. The method of claim 7, wherein blood flow status is determined by computing an energy sample value from the blood flow sample and comparing the energy sample value to a dip value and setting the energy sample value to the dip value if the energy sample value is less than the dip value.

Statement 9. The method of claim 8, wherein a blood flow conversion factor is determined based on tissue of the free tissue flap.

Statement 10. The method of claim 9, wherein determining the blood flow status of the free tissue flap and/or systemic blood flow further comprises utilizing the blood flow conversion factor.

Statement 11. The method of claim 1, wherein the quantitative blood flow status is systematic blood flow.

Statement 12. The method of claim 1, wherein the qualitative blood flow status is whether there is blood flow within a blood vessel of free tissue flap.

Statement 13. A blood flow monitor, comprising: a reference signal generator configured to generate a reference signal; a transceiver operatively connected with the reference signal generator and a probe secured to a blood vessel, the transceiver being configured to output the reference signal to the probe and receive a doppler shifted signal returned to transceiver from the probe; a signal processor operatively connected with the transceiver, the signal processor being configured to receive the doppler shifted signal from the transceiver and process the doppler shifted signal into a blood flow signal; a controller operatively connected with the signal processor, the controller being configured to receive the blood flow signal, process the blood flow signal to generate a heart rate value; and a wireless module operatively connected with the controller, the wireless module, in response to an abnormal waveform pattern, being configured to output a wireless alarm signal to a remote notification device carried by a clinician informing the clinician of an abnormal doppler shifted signal.

Statement 14. The blood flow monitor of claim 13, wherein the controller is operatively connected with the transceiver.

Statement 15. The blood flow monitor of claim 13, wherein the signal processor applied is applied on an integrated storage device or on one or more networks of remote servers.

Statement 16. The blood flow monitor of claim 15, wherein the controller determines blood flow status based at least on a blood flow sample.

Statement 17. The blood flow monitor of claim 16, wherein blood flow status is determined by computing an energy sample value of the blood flow sample and comparing the energy sample value to a dip value and setting the energy sample value to the dip value if the energy sample value is less than the dip value.

Statement 18. The blood flow monitor of claim 17, with utilizing doppler probes of a known diameter, blood flow volume within a monitored vessel is calculated. Through application of a known blood flow conversion factor, systemic hemodynamic metrics such as stroke volume and stroke volume variability will be calculated.

Statement 19. The blood flow monitor of claim 13, further comprising an audio jack configured to allow a clinician to listen to the blood flow signal.

Statement 20. The blood flow monitor of claim 13, wherein the wireless module is configured to initiate a buzzer or alarm that alerts patient and/or text message or notification to a clinician.

The preceding description provides various examples of the systems and methods of use disclosed herein which may contain different method steps and alternative combinations of components. It should be understood that, although individual examples may be discussed herein, the present disclosure covers all combinations of the disclosed examples, including, without limitation, the different component combinations, method step combinations, and properties of the system. It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the elements that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present examples are well adapted to attain the ends and advantages mentioned as well as those that are inherent therein. The particular examples disclosed above are illustrative only and may be modified and practiced in different but equivalent manners apparent to those skilled in the art having the benefit of the teachings herein. Although individual examples are discussed, the disclosure covers all combinations of all of the examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of those examples. If there is any conflict in the usages of a word or term in this specification and one or more patent(s) or other documents that may be incorporated herein by reference, the definitions that are consistent with this specification should be adopted.

What is claimed is:

1. A method comprising:
receiving a doppler shifted signal from a probe installed at least partially in a free tissue flap;
storing the doppler shifted signal in an integrated storage device or uploading the doppler shifted signal in a one or more networks of remote servers;
processing at least part of the doppler shifted signal on the integrated storage device or on the one or more networks of remote servers with a signal processor to form a blood flow sample of the free tissue flap; and
determining a qualitative blood flow status or a quantitative blood flow status of the free tissue flap with at least one blood flow sample.

2. The method of claim 1, further comprising defining a sample frequency rate, wherein the sample frequency is predetermined or manually adjusted by a clinician or any other operator.

3. The method of claim 1, further comprising applying power to a blood flow monitor.

4. The method of claim 1, wherein the doppler shifted signal is returned to a transmitter/receiver.

5. The method of claim 1, wherein the probe is any form of a doppler shifted probe, any form of a vascular probe, and/or a cook probe.

6. The method of claim 1, applying a waveform pattern recognition filter to a generated waveform produced from the doppler shifted signal and saving the waveform in a file on a integrated storage device or the one or more networks of remote servers.

7. The method of claim 1, wherein the signal processor:
receives the doppler shifted signal with a doppler receiver amplifier;
amplifies the doppler shifted signal with the doppler receiver amplifier;
multiplies a reference signal received from a reference signal generator with the doppler shifted signal to form a blood flow signal; and
samples the blood flow signal to obtain a blood flow sample.

8. The method of claim 7, wherein blood flow status is determined by computing an energy sample value from the blood flow sample and comparing the energy sample value to a dip value and setting the energy sample value to the dip value if the energy sample value is less than the dip value.

9. The method of claim 8, wherein a blood flow conversion factor is determined based on tissue of the free tissue flap.

10. The method of claim 9, wherein determining the blood flow status of the free tissue flap and/or systemic blood flow further comprises utilizing the blood flow conversion factor.

11. The method of claim 1, wherein the quantitative blood flow status is systematic blood flow.

12. The method of claim 1, wherein the qualitative blood flow status is whether there is blood flow within a blood vessel of free tissue flap.

13. A blood flow monitor, comprising:
a reference signal generator configured to generate a reference signal;
a transceiver operatively connected with the reference signal generator and a probe secured to a blood vessel, the transceiver being configured to output the reference signal to the probe and receive a doppler shifted signal returned to transceiver from the probe;
a signal processor operatively connected with the transceiver, the signal processor being configured to receive the doppler shifted signal from the transceiver and process the doppler shifted signal into a blood flow signal;
a controller operatively connected with the signal processor, the controller being configured to receive the blood flow signal, process the blood flow signal to generate a heart rate value; and
a wireless module operatively connected with the controller, the wireless module, in response to an abnormal waveform pattern, being configured to output a wireless alarm signal to a remote notification device carried by a clinician informing the clinician of an abnormal doppler shifted signal.

14. The blood flow monitor of claim 13, wherein the controller is operatively connected with the transceiver.

15. The blood flow monitor of claim 13, wherein the signal processor applied is applied on an integrated storage device or on one or more networks of remote servers.

16. The blood flow monitor of claim 15, wherein the controller determines blood flow status based at least on a blood flow sample.

17. The blood flow monitor of claim 16, wherein blood flow status is determined by computing an energy sample value of the blood flow sample and comparing the energy sample value to a dip value and setting the energy sample value to the dip value if the energy sample value is less than the dip value.

18. The blood flow monitor of claim 17, with utilizing doppler probes of a known diameter, blood flow volume within a monitored vessel is calculated. Through application of a known blood flow conversion factor, systemic hemodynamic metrics such as stroke volume and stroke volume variability will be calculated.

19. The blood flow monitor of claim 13, further comprising an audio jack configured to allow a clinician to listen to the blood flow signal.

20. The blood flow monitor of claim 13, wherein the wireless module is configured to initiate a buzzer or alarm that alerts patient and/or text message or notification to a clinician.
